(19) United States
(12) Patent Application Publication
(10) Pub. No.: US 2010/0255096 A1
(43) Pub. Date: Oct. 7, 2010

(54) Compositions And Methods For Transmucosal Delivery Of Domperidone

(76) Inventor: Craig A. Aronchick, Villanova, PA (US)

Correspondence Address: PEPPER HAMILTON LLP, 400 BERWYN PARK, 899 CASSATT ROAD, BERWYN, PA, 19312-1183, US

(21) Appl. No.: 12/597,510

(22) PCT Filed: Apr. 25, 2008
(86) PCT No.: PCT/US08/61617

§ 371 Date: Apr. 21, 2010

Related U.S. Application Data
(60) Provisional application No. 60/914,135, filed on Apr. 26, 2007.

(51) Int. Cl. A61K 31/454; C07D 401/14; A61K 9/14; A61P 1/08; A61P 15/14

(52) U.S. Cl. 424/484; 546/199; 514/322

(57) ABSTRACT

Compositions of domperidone formulated for transmucosal delivery are provided. Also provided are methods for the treatment of nausea, vomiting, and gastrointestinal motility disorders, and methods for the enhancement of breast milk production. The methods utilize domperidone compositions formulated for transmucosal administration, administered to patients in an amount effective to treat nausea, vomiting, or gastrointestinal motility disorders, or to enhance breast milk production.

### FIELD OF THE INVENTION

The invention relates generally to oral drug delivery formulations. More specifically, the invention features compositions and methods for the transmucosal administration of domperidone.

### BACKGROUND OF THE INVENTION

Various publications, including patents, published applications, technical articles and scholarly articles are cited throughout the specification. Each of these cited publications is incorporated by reference herein, in its entirety.

Domperidone is a dopamine antagonist that does not readily cross the blood-brain barrier. The primary therapeutic use for this drug is for the treatment of nausea and vomiting, including as an anti-emetic (Champion et al. (1986) CMAJ. 135:457-61). Similarly, the drug has been demonstrated to have gastroprokinetic effects and to facilitate gastrointestinal motility (Reynolds (1989) Gastroenterol. Clinics N. Am. 18:437-57). In addition, domperidone can increase the production of the hormone prolactin, and thus can enhance lactation.

The drug is approved and administered throughout the world for various indications. In the United States, however, the Food and Drug Administration has not yet approved domperidone for any indications possibly due to an apparent risk of cardiac arrhythmias, in particular, prolongation of the QT interval. Deaths from domperidone use have been reported (Giaccone G et al. (1984) Lancet 2:1336-7).

The QT interval on the body surface electrocardiogram (ECG) is a measurement of ventricular repolarization as determined by transmembrane action potential duration of the ventricular myocardium. Delayed ventricular repolarization that manifests as QT interval prolongation on the ECG is associated with the development of an atypical form of polymorphic ventricular tachycardia termed torsade de pointes (TdP) that can result in recurrent fainting and sudden death in humans. Many drugs have been pulled from distribution, and lead compounds pulled from further development due to the risk of QT prolongation. For example, cisapride, a drug used for proximal gastrointestinal motility disorders, was pulled from the market in 2000 because of its arrhythmogenic potential and sudden death (Drolet et al. (1998) Circulation 97:204-10).

In the case of domperidone, cardiac arrhythmias and death are believed, by some, to be confined to cases where the patient received high doses of the drug intravenously, or otherwise had risk factors for such adverse effects (Osborne et al. (1985) Hum. Toxicol. 4:617-26). Nevertheless, the severe nature of these potential side effects has contributed to disapproval of the manufacture, marketing, or dispensation of this drug in the United States.

There is a need for antiemetic and promotility drugs that provide low toxicity and few harmful side effects. Given the many beneficial effects domperidone has to offer, there is a need to develop and provide new formulations that provide rapid and significant bioavailability of the drug, allowing for lower doses to produce the beneficial effect with a concomitant reduction in the risk of adverse cardiac events. The present invention addresses these and other needs.

### SUMMARY OF THE INVENTION

In one embodiment, the invention relates to compositions comprising domperidone for transmucosal delivery. Compositions are suitable for oral transmucosal use, and provide for absorption of the drug across the oral mucosa.

The invention is also directed to methods of treatment comprising administering domperidone to a patient by transmucosal delivery. The inventive methods may improve bioavailability relative to oral dosage forms, especially in those patients with abnormally slow gastric emptying. Such methods can involve administration of the novel domperidone-containing compositions described herein. The methods may provide treatment for a variety of conditions amenable to amelioration by domperidone administration, without the occurrence of side effects, such as QT interval prolongation, that have resulted from prior art treatment methods involving the drug (Medical Control Council (2006) Pharmacovigilance).

The invention is directed to a pharmaceutical composition comprising from about 1 mg to about 9 mg of domperidone. In some embodiments, the pharmaceutical composition comprises from about 2 mg to about 4 mg of domperidone.

The invention is directed to pharmaceutical compositions comprising from about 1 mg to about 9 mg of domperidone wherein the composition comprises a permeation enhancer. In some embodiments, the permeation enhancer is chosen from: a bile salt, sodium dodecyl sulfate, dimethyl sulfoxide, sodium lauryl sulfate, a derivative of a saturated or a unsaturated fatty acid, a surfactant, a bile salt analog, and a derivative of a bile salt. In some embodiments the permeation enhancer is a synthetic permeation enhancer.

In some embodiments, the pharmaceutical composition comprising domperidone further comprises at least one flavoring agent, artificial coloring, sweetener, lubricating agent, disintegration agent, lubricating agent, diluent, base, or buffering agent. In some embodiments, the pharmaceutical composition comprising domperidone further comprises a sustained release agent.

In some embodiments the pharmaceutical composition comprises a concentration of domperidone is from about 0.01% to about 90% of the dry matter weight of the composition. In some embodiments, the pharmaceutical composition comprises domperidone that is covalently bonded to a chemical modifier. In some embodiments, the domperidone is incorporated into a dissolvable matrix material.

The invention is directed to an oral transmucosal solid dosage form comprising from about 1 mg to about 9 mg of domperidone. In some embodiments, the oral transmucosal solid dosage form comprises from about 2 mg to about 4 mg of domperidone.

In some embodiments, the oral transmucosal solid dosage further comprises a permeation enhancer. In some embodiments, the permeation enhancer is chosen from: a bile salt, sodium dodecyl sulfate, dimethyl sulfoxide, sodium lauryl sulfate, a derivative of a saturated or a unsaturated fatty acid, a surfactant, a bile salt analog, and a derivative of a bile salt. In some embodiments the oral transmucosal dosage form is chosen from: a chewing gum, a patch, a lozenge, a lozenge-on-a-handle, a tablet, a troche, a pastille, a sachet, a sublingual tablet, and a rapid disintegrating tablet. In some embodiments, the oral transmucosal solid dosage form of wherein the composition further comprises at least one flavoring agent, artificial coloring, sweetener, lubricating agent, disintegration agent, lubricating agent, diluent, base, or buffering agent. In some embodiments, the oral transmucosal solid dosage form further comprises a sustained release agent.

The invention is directed to an oral transmucosal solid dosage form comprising from wherein the concentration of domperidone is from about 0.01% to about 90% of the dry matter weight of the composition.

In some embodiments of the oral dosage form, domperidone is covalently bonded to a chemical modifier. In some embodiments of the oral dosage form, domperidone is incorporated into a dissolvable matrix material.

The invention relates to a method of delivering a pharmaceutical composition comprising from about 1 mg to about 9 mg of domperidone into a subject by transmucosal administration.

In some embodiments of the method, the pharmaceutical composition is administered orally, intranasally, vaginally, rectally, or transdermally. In some embodiments, at least one other therapeutic agent is co-administered with the pharmaceutical composition comprising from about 1 mg to about 9 mg of domperidone. In some embodiments, administration of the pharmaceutical composition of is sublingual.

In some embodiments the method includes administration of a pharmaceutical composition comprising a concentration of domperidone is from about 0.01% to about 90% of the dry matter weight of the composition. In some embodiments, includes administration of a pharmaceutical composition comprising a domperidone wherein the subject is administered a total amount of from about 1 mg to about 500 mg of domperidone per day. In some embodiments, includes administration of a pharmaceutical composition comprising a domperidone wherein the subject is administered a total amount of up to about 40 mg of domperidone per day.

A method of treating nausea or vomiting in a subject by transmucosal administration of a therapeutically effective amount of the pharmaceutical composition comprising domperidone to the subject. In some embodiments, the method of treating nausea or vomiting in a subject comprises the pharmaceutical composition being administered intranasally, vaginally, rectally, orally, or transdermally. In some embodiments, the method of treating nausea or vomiting in a subject comprises the pharmaceutical composition being administered orally. In some embodiments, the method of treating nausea or vomiting in a subject comprises the pharmaceutical composition being administered sublingually. In some embodiments, the method of treating nausea or vomiting in a subject comprises co-administration of the pharmaceutical composition comprising domperidone and at least one other therapeutic agent. In some embodiments, the method of treating nausea or vomiting in a subject comprises administration of the pharmaceutical composition comprising domperidone in a daily dose range of from about 0.01 mg/kg to about 500 mg/kg of domperidone relative to the weight of the subject. In some embodiments, the method of treating nausea or vomiting in a subject comprises administration of a total amount of from about 1 mg to about 500 mg of domperidone per day. In some embodiments, the method of treating nausea or vomiting in a subject comprises administration of up to about 40 mg of domperidone per day.

The invention relates to a method of treating a gastrointestinal motility disorder in a subject by transmucosal administration of a therapeutically effective amount of the pharmaceutical composition comprising from about 1 mg to about 9 mg of domperidone into a subject. In some embodiments, the method of treating a gastrointestinal motility disorder in a subject comprises the pharmaceutical composition being administered intranasally, vaginally, rectally, orally, or transdermally. In some embodiments, the method of treating treating a gastrointestinal motility disorder in a subject comprises the pharmaceutical composition being administered orally. In some embodiments, the method of treating a gastrointestinal motility disorder in a subject comprises the pharmaceutical composition being administered sublingually. In some embodiments, the method of treating a gastrointestinal motility disorder in a subject comprises co-administration of the pharmaceutical composition comprising domperidone and at least one other therapeutic agent. In some embodiments, the method of treating a gastrointestinal motility disorder in a subject comprises administration of the pharmaceutical composition comprising domperidone in a daily dose range of from about 0.01 mg/kg to about 500 mg/kg of domperidone relative to the weight of the subject. In some embodiments, the method of treating a gastrointestinal motility disorder in a subject comprises administration of a total amount of from about 1 mg to about 500 mg of domperidone per day. In some embodiments, the method of treating a gastrointestinal motility disorder in a subject comprises administration of up to about 40 mg of domperidone per day.

The invention relates to enhancing lactation in a subject by transmucosal administration of a therapeutically effective amount of the pharmaceutical composition comprising from about 1 mg to about 9 mg of domperidone into a subject. In some embodiments, the method of enhancing lactation in a subject comprises the pharmaceutical composition being administered intranasally, vaginally, rectally, orally, or transdermally. In some embodiments, the method of enhancing lactation in a subject comprises the pharmaceutical composition being administered orally. In some embodiments, the method of enhancing lactation in a subject comprises the pharmaceutical composition being administered sublingually. In some embodiments, the method of enhancing lactation in a subject comprises co-administration of the pharmaceutical composition comprising domperidone and at least one other therapeutic agent. In some embodiments, the method of enhancing lactation in a subject comprises administration of the pharmaceutical composition comprising domperidone in a daily dose range of from about 0.01 mg/kg to about 500 mg/kg of domperidone relative to the weight of the subject. In some embodiments, the method of enhancing lactation in a subject comprises administration of a total amount of from about 1 mg to about 500 mg of domperidone per day. In some embodiments, the method of enhancing lactation in a subject comprises administration of up to about 40 mg of domperidone per day.

### DETAILED DESCRIPTION OF ILLUSTRATIVE EMBODIMENTS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used in this specification and the appended claims, the singular forms “a,” “an,” and “the” include plural referents unless the content clearly dictates otherwise.

The term “about” as used herein when referring to a measurable value such as an amount, a temporal duration, and the like, is meant to encompass variations of ±20%. Alternatively, the term “about” is meant to encompass ±10%, ±5%, ±1%, or ±0.1% from the specified value, as such variations are appropriate to perform the disclosed methods.

The present invention is directed to compositions and methods for the transmucosal administration of domperidone. “Domperidone” refers to the compound: 5-chloro-1-[1-[3-(2-oxo1,3-dihydrobenzoimidazol-1-yl)propyl]-4-piperidyl]1,3-dihydrobenzoimidazol-2-one, and has the following structure:

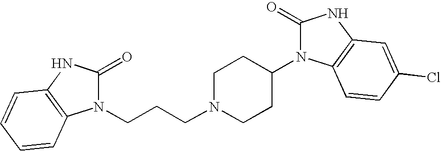

In accordance with the present invention, domperidone can be formulated into delivery systems for release of the drug across a mucosal membrane. Such transmucosal formulations may provide rapid and substantial bioavailability of domperidone by avoiding the destructive acidic environment of the stomach, avoiding variability in delayed gastric emptying, and avoiding first pass metabolism by the liver. The inventive formulations may provide the additional advantage of substantially reducing the risk that a subject to which the formulations are administered will develop a cardiac arrhythmia, since total doses administered for efficacy will likely be less due to enhanced absorption. Accordingly, the invention features compositions comprising domperidone that are formulated for transmucosal delivery.

Solid pharmaceutical dosage forms are well known in the art. Oral solid dosage forms are suitable dosage forms and account for eighty percent of all the pharmaceutical products on the market. Solid dosage forms are easier for a patient or caregiver to identify, handle and administer. They are also non-invasive and have high patient compliance.

Solid dosage forms can be further divided into several groups, based upon the route by which the drug is delivered, including, for example, gastrointestinal (GI) tract delivery, suppository (rectal, vaginal and urethral) delivery, transdermal delivery and oral transmucosal delivery. The majority of solid dosage forms on the market are designed for gastro-intestinal delivery. GI delivery is often referred to simply as “oral delivery,” because a tablet or capsule is initially introduced orally, and swallowed. However, this type of solid delivery form is designed to dissolve in the GI tract, where absorption of the drug occurs. Solids are also commonly delivered as suppositories such as laxatives, contraceptives and hemorrhoid medication. Relatively few drug formulations are designed as solid dosage forms intended to deliver a drug through the oral mucosa.

Despite the overall popularity of other delivery methods, oral transmucosal (OT) delivery is a particularly advantageous delivery route. One of the advantages of OT delivery is that it is non-invasive. Furthermore, OT delivery generally has better patient compliance, less risk of infection and lower cost than invasive procedures such as injection and implantation. It also has much shorter onset time, i.e., the time from administration to therapeutic effect, than does oral delivery. A drug absorbed via the oral mucosa will also avoid first pass metabolism, in which the drug is metabolized in the GI tract and liver. Similarly, a drug absorbed via the oral mucosa avoids the variability in gastric emptying time commonly observed in patients with proximal gastrointestinal motility syndromes, allowing for greater predictability in obtaining therapeutic blood levels. Oral transmucosal delivery is simple and oral transmucosal dosage forms can be administered by the caregiver or the patient with minimal discomfort.

The absorption of a drug across the mucosal tissue can be described using an expression based on Fick's law of diffusion:

$$\frac{d A}{d t} = \frac{{DK}_{p}}{h} \cdot ( {C}_{1} - {C}_{2} ) \cdot S$$

where dA is the amount of drug delivered over time dt, D is the diffusion coefficient of the drug inside the oral mucosal tissue, Kₚ is the partition coefficient of the drug between oral mucosal tissue and the drug solution, S is the surface area of the oral cavity, h is the thickness of the oral mucosal tissue, and C₁ and C₂ are the drug concentrations at the absorption site and in blood, respectively.

The capacity of oral transmucosal drug delivery is limited in large part by the surface area available for drug absorption. The surface area in the oral cavity is approximately 200 cm², which is relatively small compared to the surface area of other drug delivery routes, such as the GI tract (350,000 cm²) and skin (20,000 cm²).

The contact time between the drug and the absorption surface is primarily controlled by the dissolution rate of the solid unit. Once an immediate release solid unit is dissolved, any drug solution not yet absorbed will generally be swallowed shortly thereafter, thereby ending further OT drug absorption. Such immediate release dissolvable dosage units are usually designated as “open” delivery systems. A solid dosage unit can be designed to remain in the oral cavity for different periods of time. Generally, for rapid onset of effect, a solid unit is designed to remain in the oral cavity for about 10 to 15 minutes or less, when used as directed.

In addition to the difficulties presented by the oral cavity's unique environment, the physicochemical properties of the drug can present challenges and complications that affect oral transmucosal drug delivery. Primarily, the solubility, the dissolution rate, and the partition coefficient determine the extent to which a drug can be delivered via the oral mucosal tissue. Solubility of the drug and/or bulking agent can be a rate limiting step. Solubility and dissolution rate are key aspects in creating the concentration gradient, which is the driving force for drug delivery. Partition coefficient, on the other hand, acts like an amplifier, such that the drug delivery rate is directly proportional to the partition coefficient up to a point.

Various solid dosage forms may be used to deliver drugs via the oral mucosal tissue. The chewing gum delivery dosage form, for example, may be useful for patients who may be more disposed to self-administer a drug in a chewing gum form as opposed to other less familiar dosage forms. The gum may also be used to mask the taste of various pharmaceutical ingredients. The gum matrix in the formulation may also be designed to extend the duration of drug delivery.

Transmucosal delivery of drugs may also be accomplished through the use of patches which are attached to the oral mucosa by a bio-adhesive. Buccal patches are generally designed as a “closed” delivery system, that is, the environmental conditions inside the patch are primarily controlled by the formulation. Employing a closed delivery system can facilitate drug delivery, such as allowing the use of enhancers or other permeability facilitators in the formulation which might otherwise be impractical.

Solid dosage forms such as lozenges and tablets may also be used for oral transmucosal delivery of pharmaceuticals. For example, nitroglycerin sublingual tablets have been on the market for many years. The sublingual tablets are designed to deliver small amounts of the potent nitroglycerin, which is almost immediately dissolved and absorbed. On the other hand, most lozenges or tablets are typically designed to dissolve in the mouth over a period of at least several minutes which allows extended dissolution of the lozenge and absorption of the drug.

Administration of lozenges or sublingual tablets generally utilize an “open” delivery system, in which the drug delivery conditions are influenced by the conditions of the surrounding environment, such as rate of saliva secretion, pH of the saliva, or other conditions beyond the control of the formulation.

A lozenge-on-a-handle (similar to a lollipop) is another dosage form suitable for transmucosal drug delivery. In addition to being non-invasive and providing a particularly easy method of delivery, the lozenge-on-a-handle (or lozenge with an integrated oral transmucosal applicator) dosage form allows a patient or caregiver to move the dosage form in and out of the mouth to titrate the dose. This practice is called dose-to-effect, in which a patient or caregiver controls the administration of the dose until the expected therapeutic effect is achieved. This is particularly important for certain symptoms, such as pain, nausea, motion sickness, and premedication prior to anesthesia because each patient needs a different amount of medication to treat these symptoms. For these types of treatments, the patient is the only one who knows how much medication is enough. Once the appropriate amount of drug is delivered, the patient or caregiver can remove the lozenge-on-a-handle, thus, stopping delivery of the drug. This feature is especially important for particularly potent drugs, which may present a significant advantage of terminating drug administration once the desired effect is achieved.

Other suitable oral transmucosal dosage forms may include rapid disintegrating tablets, as described, for example, in U.S. Pat. No. 6,696,085, to Rault, et al., the disclosure of which is incorporated herein by reference in its entirety. Such tablets make use of a disintegrating agent, such as a methacrylic acid copolymer, to provide rapid tablet dissolution, leading to rapid transmucosal absorption.

As used herein, the term “oral transmucosal delivery” (OTD) refers to the delivery of a pharmaceutical agent across a mucous membrane in the oral cavity, pharyngeal cavity, or esophagus, and may be contrasted, for example, with traditional oral delivery, in which absorption of the drug occurs in the intestines. Accordingly, routes of administration in which the pharmaceutical agent is absorbed through the buccal, sublingual, gingival, pharyngeal, and/or esophageal mucosa are all encompassed within “oral transmucosal delivery,” as that term is used herein. Oral transmucosal delivery involves the administration of an oral transmucosal solid dosage form to the oral cavity of a patient, which is held in the oral cavity and dissolved, thereby releasing the pharmaceutical agent for oral transmucosal delivery. Of course, as the solid dosage form dissolves in the oral cavity, some of the saliva containing the pharmaceutical agent may be swallowed, and a portion of the drug may ultimately be absorbed from the intestines.

As used herein, the term “oral transmucosal solid dosage form” broadly refers to any solid delivery form suitable for administering a pharmaceutical agent by oral transmucosal delivery, including patches, troches, lozenges, pastilles, sachets, sublingual tablets, lozenges-on-a-handle (otherwise referred to as lollipops), rapid disintegrating tablets, and the like. Forms of oral transmucosal solid dosage forms include patches, lozenges, sublingual tablets, and rapid disintegrating tablets. The solid dosage form may be held between the cheek and gum or placed on or under the tongue, or it may be actively licked, sucked, or rubbed across the oral mucosa by the patient or a caregiver. In some embodiments, the solid dosage form is not bitten or chewed, unless the broken pieces are then held in the mouth until substantially dissolved.

Employing the pharmaceutical compositions of the present invention, a pharmaceutical agent may be introduced into the patient's bloodstream almost as fast as through injection, and much faster than using the oral administration route, while avoiding the negative aspects of those delivery methods. The present invention may achieve these advantages by incorporating the drug into a dissolvable matrix material. A solid dosage form within the scope of the present invention can be used to administer drugs in a dose-to-effect manner, or until the precise desired effect is achieved.

As noted previously, however, an oral transmucosal solid dosage form ideally exhibits satisfactory patient-controlled dissolution rates, drug stability, and otherwise be suitable for oral transmucosal delivery. In order to meet these criteria, it is generally desired to disperse the pharmaceutical agent in a pharmaceutically acceptable excipient, that provides both bulk, and helps control the dissolution rate. “Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered. The term “patient,” as used herein, refers to animals, including mammals, preferably humans.

In addition to domperidone, the compositions of the present invention may also contain optional ingredients, such as flavorings, sweeteners, flavor enhancers, releasing or lubricating agents, and permeation enhancers. The term “permeation enhancer” as used herein refers to any agent that increases permeability of a mucosa to an active agent, or otherwise facilitates penetration of the drug through a mucosa. In some embodiments, these inactive ingredients shall be on the GRAS list (“generally regarded as safe”), to assure that they are pharmaceutically acceptable. Alternatively, an inactive ingredient should be self proclaimed GRAS or, at least, acceptable in food.

It will be appreciated that a change of the pH may also change the taste characteristics of the drug. Drugs which are very high in pH typically are very bitter in taste. As the pH drops, the taste becomes less bitter, then salty, and may eventually become sour. Flavorings can more adequately improve the taste characteristics of drugs in the lower pH ranges. As a result, in addition to impacting the bioavailability, buffering pH may also affect the taste characteristics of the composition.

Nonetheless, it may be desirable to add a flavoring agent to the compositions of the present invention. A wide range of flavors are available for preparing good tasting and desirable medications within the scope of the present invention. These may be added to mask the taste of the drug. Flavorings may be combined, as desired, to produce a particular flavor mix which is compatible with a particular medication. Some of the confectioner's flavorings which may be used in the context of the present invention include artificial vanilla, vanilla cream, mint, berry, cherry, spearmint, grape, coconut, chocolate, menthol, licorice, lemon, and butterscotch. Each of these flavorings is obtainable in a concentrated powder form. Sin some embodments, flavoring agents are prepared by spray drying. Other flavorings known in the confectionary arts may be acceptable because of the ease of combining the ingredients of the present invention. Any number of flavorings may be combined in any desired ratio to produce the specific desired taste characteristics required for any particular application. For example, flavor combinations may be varied in order to be compatible with the flavor characteristics of any specific drug.

To produce a desirable color for the end product, artificial colorings may also be added to the composition. The flavorings described above are generally a white powder, as are the other major components. Therefore, additional coloring maybe necessary if a colored end product is desired. Coloring may also be used as a code to indicate the type and concentration of drug contained within a particular lozenge-on-a-handle. Any type of color known to be “generally regarded as safe” (“GRAS”), and thus generally used in the confectionary trade, or otherwise approved by the appropriate regulatory authority for use in pharmaceutical preparations, may be used to provide coloring to the product.

To provide a good tasting medication, it may be desired to add sugars, sugar alcohols, or other sweeteners to the composition. Suitable artificial sweeteners include, but are not limited to, aspartame, acesulfame K, saccharin, sucralose, altitame, cyclamic acid and its salts, glycerrhizinate, dihydrochalcones, thaumatin, monellin, or any other non-cariogenic, sugar-free sweetener, alone or in combination. For compositions which contain a sugar alcohol based excipient, additional sweeteners may not be desired, due to the naturally sweet taste of these polyhydric alcohols. It is desired that a sweetener or combination of sweeteners be obtained which is compatible with the pharmaceutical agent and the other components such that a good tasting solid dosage form is produced.

For some applications, it may be desirable to add a flavor enhancer to the composition to achieve a good tasting product. Flavor enhancers may provide a more pleasant sensation in the patient's mouth during oral transmucosal administration. Flavor enhancers within the scope of the present invention include materials such as ribotide (a nucleotide) and monosodium glutamate (“msg”). Other flavor enhancers are known to those of skill in the art.

Most drugs are present in solution in both the unionized and ionized forms. Generally lipid soluble or lipophilic drugs diffuse most readily across mucosal membranes. Accordingly, the compositions of the present invention may contain buffering agents, pH-adjusting agents, or ionizing agents to adjust the ratio of unionized:ionized forms of the domperidone.

Whether a drug exists in the ionized or non-ionized (unionized) form is largely dependent upon its pKa, and correspondingly on the pH of the solution in which it is dispersed. pKa is defined as the negative logarithm (base 10) of the dissociation constant (Ka). pKa may also be defined as the pH at which a given acid or base is 50% ionized and 50% unionized. Using the well-known Henderson-Hasselbalch equation, concentrations of the charged and uncharged species of a drug can easily be calculated, if the pKa and pH are known. From that equation, it is clear that the ionized portion of the drug will be decreased by lowering the pH for weak acid drugs and increasing the pH for drugs that are weak bases. Domperidone has a pKa of 7.9. Thus, adding an ionizing agent that maintains a more acidic pH will increase the portion of domperidone that is present in the ionized form, which may lead to a decrease in the oral transmucosal absorption, and hence, bioavailability. Conversely, adding an agent (e.g., a base or buffer) that maintains a pH close to 7.9 will lead to in increase in the percentage of the drug that is present in the unionized form, and potentially increase bioavailability.

Domperidone diffusion across mucosal membranes may also be improved if the mucosal membrane is treated with a permeation enhancer. It has also been found that certain permeation enhancers can significantly enhance the permeability of lipophilic and nonlipophilic drugs. Other penetration enhancers are described in Cooper et al. (1987) “Penetration Enhancers”, in Transdermal Delivery of Drugs, Vol. II, Kyodonieus et al., Eds., CRC Press, Boca Raton, Fla. Not all pharmaceutical agents fit this profile, however, and those which do are not always predictably absorbed. Additional forms of chemical enhancers, such as those enhancing lipophilicity, have been developed to improve transport when physically mixed with certain therapeutic agents and provide more predictable absorption. See for example, U.S. Pat. Nos. 4,645,502; 4,788,062; 4,816,258; 4,900,555; 3,472,931; 4,006,218; and 5,053,227. Carriers have also been coupled to pharmaceutical agents to enhance intracellular transport. See Ames et al. (1973) Proc. Natl. Acad. Sci. USA, 70:456-458 and (1988) Proc. Int. Symp. Cont. Rel. Bioact. Mater., 15:142.

Typical permeation enhancers may include bile salts such as sodium cholate, sodium glycocholate, sodium glycodeoxycholate, taurodeoxycholate, sodium deoxycholate, sodium lithocholate chenocholate, chenodeoxycholate, ursocholate, ursodeoxycholate, hydrodeoxycholate, dehydrocholate, glycochenocholate, taurochenocholate, and taurochenodeoxycholate. Other permeation enhancers such as sodium dodecyl sulfate (“SDS”), dimethyl sulfoxide (“DMSO”), sodium lauryl sulfate, salts and other derivatives of saturated and unsaturated fatty acids, surfactants, bile salt analogs, derivatives of bile salts, or such synthetic permeation enhancers as described in U.S. Pat. No. 4,746,508, the disclosure of which is incorporated herein by reference in its entirety, may also be used. It is generally believed that bile salts are good enhancers for hydrophilic drugs and long chain fatty acids, their salts, derivatives, and analogs are more suitable for lipophilic drugs. DMSO, SDS, and medium chain fatty acids (about C-8 to about C-14) their salts, derivatives, and analogs may work for both hydrophilic and lipophilic drugs.

The permeation enhancer concentration within the dissolvable matrix material may be varied depending on the potency of the enhancer and rate of dissolution of the dissolvable matrix. Other criteria for determining the enhancer concentration include the potency of the drug and the desired lag time. The upper limit for enhancer concentration is set by toxic effect to or irritation limits of the mucosal membrane.

The following is a list of typical enhancers and an exemplary concentration range for each enhancer:

TABLE 1

| | Operational | Preferred |
|---|---|---|
| Enhancer | Concentration | Range |
| sodium cholate | 0.02%-50% | 0.1%-16% |
| sodium dodecyl sulfate | 0.02%-50% | 0.1%-2% |
| sodium deoxycholate | 0.02%-50% | 0.1%-16% |
| taurodeoxycholate | 0.02%-solubility | 0.1%-16% |
| sodium glycocholate | 0.02%-solubility | 0.1%-16% |
| sodium taurocholate | 0.02%-solubility | 0.1%-16% |
| DMSO | 0.02%-solubility | 5%-50% |

An alternative method of increasing transmucosal delivery, described, for example, in Hale, et al., U.S. Pat. No. 5,607,691, involves covalently bonding the pharmaceutical agent to a chemical modifier, via a physiologically cleavable bond, such that the membrane transport and delivery of the agent is enhanced.

Additional methods of increasing transmucosal delivery involves the use of a permeation enhancer in combination with a C2 and C4 alkanol and a polyalcohol as described in US Patent Application 20070166361 (based upon PCT/EP00/07533).

Additional methods of increasing transmucosal delivery involves the use of a permeation enhancer mixture in combination with a C2 and C4 alkanol, a polyalcohol, and water as described in US Patent Application 20070098775.

In certain medications, it may also be desirable to add a lubricating agent in order to facilitate the manufacturing process. Such agents may also provide a certain amount of waterproofing. The rate of dissolution of the solid dosage form within the patient's mouth may be controlled chemically, as well as physically, for example, through the extent of compression of the composition (where the product is a compressed powder solid dosage form). These lubricating or releasing agents may include substances such as compritol 888, calcium stearate, and sodium stearate. These agents may enhance dissolution or they may inhibit dissolution as necessary.

As mentioned previously, the compositions may also include a disintegrating agent. Tablet disintegrators are substances which swell when-wetted to break up the tablet and release the compound, and include, but are not limited to, starches such as corn and potato starches, clays, celluloses, aligns, gums, methylcellulose, agar, bentonite, wood cellulose, powdered natural sponge, cation-exchange resins, alginic acid, guar gum, citrus pulp, carboxymethyl cellulose, and sodium lauryl sulfate. Acrylic type polymers can also advantageously be used as disintegrators, including methacrylic copolymers of type C (as disclosed in U.S. Pat. No. 6,696,085).

Solid forms can be prepared according to any means suitable in the art. For example, capsules are prepared by mixing the domperidone composition with a suitable diluent and filling the proper amount of the mixture in capsules. Tablets are prepared by direct compression, by wet granulation, or by dry granulation. Their formulations usually incorporate diluents, binders, lubricants and disintegrators as well as the compound. Diluents, but are not limited to, include various types of starch, cellulose, crystalline cellulose, microcrystalline cellulose, lactose, fructose, sucrose, mannitol or other sugar alcohols, kaolin, calcium phosphate or sulfate, inorganic salts such as sodium chloride and powdered sugar. Powdered cellulose derivatives are also useful. Non-limiting examples of tablet binders include, but are not limited to, starches, gelatin and sugars such as lactose, fructose, glucose and the like. Natural and synthetic gums are also convenient, including, but are not limited to, acacia, alginates, methylcellulose, polyvinylpyrrolidone and the like. Polyethylene glycol, ethylcellulose and waxes can also serve as binders.

A lubricant can be used in a tablet formulation to prevent the tablet and punches from sticking in the die. The lubricant include, but are not limited to, such slippery solids as talc, magnesium and calcium stearate, stearic acid and hydrogenated vegetable oils.

Tablets can be coated with sugar as a flavor and sealant, or with film-forming protecting agents to modify the dissolution properties of the tablet. The compounds may also be formulated as chewable tablets, by using large amounts of pleasant-tasting substances such as mannitol in the formulation, as is now well-established in the art.

Also contemplated are liquid formulations and solid form preparations which are intended to be converted, shortly before use, to liquid form preparations. Such liquid forms include, but are not limited to, solutions, suspensions, syrups, slurries, and emulsions. Liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). These preparations may contain, in addition to the active agent, colorants, flavors, stabilizers, buffers, artificial and natural sweeteners, dispersants, thickeners, solubilizing agents, and the like. The compositions may be in powder form for constitution with a suitable vehicle such as sterile water, saline solution, or alcohol, before use. Preparations may also contain mucosal enhancers.

The domperidone compositions can be administered intranasally, vaginally, rectally, orally, or transdermally. In some embodiments, the compositions are administered orally. Administration may be at the direction of a physician.

For buccal and sublingual administration the compositions may take the form of tablets, troche or lozenge formulated in conventional manner, as described above, or a suspension that is retained. Compositions for oral or buccal administration, may be formulated to give immediate or controlled release of the active compound. Such formulations may include one or more sustained-release agents known in the art, such as glyceryl mono-stearate, glyceryl distearate and wax.

Also featured in accordance with the present invention are methods of treatment that utilize the inventive domperidone formulations. The terms “treating” or “treatment” refer to any success or indicia of success in the attenuation or amelioration of an injury, pathology, disease, or condition, including any objective or subjective parameter such as abatement, remission, diminishing of symptoms or making the injury, pathology, disease, or condition more tolerable to the patient, slowing in the rate of degeneration or decline, improving a subject's physical or mental well-being, or prolonging the length of survival. The treatment or amelioration of symptoms can be based on objective or subjective parameters; including the results of a physical examination, neurological examination, and/or psychiatric evaluations.

“Effective amount” or “therapeutically effective amount” are used interchangeably herein, and refer to an amount of a compound, material, or composition, as described herein effective to achieve a particular biological result such as, but not limited to, biological results disclosed, described, or exemplified herein. Such results may include, but are not limited to, the suppression of nausea or vomiting, or the enhancement or promotion of lactation, as determined by any means suitable in the art.

One aspect features methods for treating nausea in a subject. Another aspect features methods for treating vomiting in a subject. Another aspect features methods for treating gastrointestinal motility disorders in a subject. Another aspect features methods for enhancing lactation in a subject. In one embodiment, a subject with nausea can be identified and treated with a transmucosal formulation of domperidone as described herein. In another embodiment, a subject with a gastrointestinal motility disorder can be identified and treated with a transmucosal formulation of domperidone as described herein. In another embodiment, a subject in need of enhanced breast milk production can be identified and treated with a transmucosal formulation of domperidone as described herein.

The inventive methods are amenable to treating nausea of any origin, such as nausea induced by motion sickness, by a reaction to administration of a drug, including chemotherapy regimens, by pregnancy, and the like. The nausea can be short-term, or long-term. Nausea can be accompanied by vomiting. The inventive methods are amenable to treating gastrointestinal motility disorders of any type, including functional disorders such as Irritable Bowel Syndrome, constipation, Gastroparesis, Hirschsprung's disease, chronic intestinal pseudo-obstruction, Achalasia, and the like. The inventive methods can be utilized to treat proximal gastrointestinal motility disorders such as gastroparesis, functional dyspepsia, and gastroesophageal reflux disease.

To treat a subject afflicted with nausea, vomiting, gastrointestinal motility disorders, or insufficient breast milk production, a therapeutically effective amount of domperidone appropriate for the disorder being treated is administered to the subject. A therapeutically effective amount may provide a clinically significant reduction in the subject's nausea and/or vomiting, enhancement in gastrointestinal motility, for example, an increase in esophageal peristalsis, lower esophageal sphincter pressure, gastric motility, or peristalsis, enhancement of gastroduodenal coordination, or facilitation of gastric emptying, or an enhancement of breast milk production/lactation, and the like.

The effective amount of the composition may be dependent on any number of variables, including, without limitation, the species, breed, size, height, weight, age, overall health of the subject, the type of formulation, the mode or manner or administration, the type and/or severity of the particular condition being treated, or the need to enhance lactation. The appropriate effective amount can be routinely determined by those of skill in the art using routine optimization techniques and the skilled and informed judgment of the practitioner and other factors evident to those skilled in the art. In some embodiments, a therapeutically effective dose of the compounds described herein may provide therapeutic benefit without causing substantial toxicity to the subject.

Toxicity and therapeutic efficacy of the inventive domperidone formulations can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents or compositions which exhibit large therapeutic indices are preferred. The data obtained from cell culture assays and animal studies can be used in formulating a range of dosage for use in the subject. The dosage of such agents or compositions lies preferably within a range of circulating concentrations that include the ED₅₀ with little or no toxicity. The dosage may vary within this range depending upon the dosage form employed and the route of administration utilized.

For any domperidone composition used in the methods of the invention, the therapeutically effective dose can be estimated initially from in vitro assays such as cell culture assays. For example, a dose can be formulated in animal models to achieve a circulating plasma concentration range that includes the IC₅₀ as determined in cell culture (e.g., the concentration of the composition which achieves a half-maximal suppression of nausea or vomiting). Such information can be used to more accurately determine useful doses in a specified subject such as a human. The treating physician can terminate, interrupt, or adjust administration due to toxicity, or to organ dysfunctions, and can adjust treatment as necessary if the clinical response is not adequate in order to improve the response.

In one aspect of the inventive methods, the compositions comprise a concentration of domperidone in a range of from about 0.01% to about 90% of the dry matter weight of the composition. In some embodiments, domperidone comprises up to about 75% of the dry matter weight of the composition. In some embodiments, domperidone comprises up to about 50% of the dry matter weight of the composition. In some embodiments, domperidone comprises up to about 40% of the dry matter weight of the composition. In some embodiments, domperidone comprises up to about 30% of the dry matter weight of the composition. In some embodiments, domperidone comprises up to about 25% of the dry matter weight of the composition. In some embodiments, domperidone comprises up to about 20% of the dry matter weight of the composition. In some embodiments domperidone comprises up to about 15% of the dry matter weight of the composition. In some embodiments, domperidone comprises up to about 10% of the dry matter weight of the composition.

In some embodiments, subjects can be administered domperidone formulations in which domperidone is provided in a daily dose range of from about 0.01 mg/kg to about 500 mg/kg of the weight of the subject. The dose administered to the subject can also be measured in terms of total amount of drug administered per day. Dosing regimens of OTD-domperidone may range from as low as 1 mg four times per day to as high as 30 mg four times per day. In some embodiments, dosing regimens may be about 2 mg administered four times a day. In some embodiments, dosing regimens may be about 3 mg administered four times a day. In some embodiments, dosing regimens may be about 4 mg administered four times a day. In some embodiments, dosing regimens may be about 5 mg administered four times a day. In some embodiments, dosing regimens may be about 6 mg administered four times a day. In some embodiments, dosing regimens may be about 7 mg administered four times a day. In some embodiments, dosing regimens may be about 8 mg administered four times a day. In some embodiments, dosing regimens may be about 9 mg administered four times a day. In some embodiments, dosing regimens may be about 10 mg administered four times a day. In some embodiments, a subject is administered from about 1 to about 500 milligrams of domperidone per day. In some embodiments, a subject is administered up to about 10 milligrams of domperidone per day. In some embodiments, a subject is administered up to about 20 milligrams of domperidone per day. In some embodiments, a subject is administered up to about 30 milligrams of domperidone per day. In some embodiments, a subject is administered up to about 40 milligrams of domperidone per day. In some embodiments, a subject is administered up to about 50 milligrams of domperidone per day. In some embodiments, a subject is administered up to about 60 milligrams of domperidone per day. In some embodiments, a subject is administered up to about 70 milligrams of domperidone per day. In some embodiments, a subject is administered up to about 80 milligrams of domperidone per day. In some embodiments, a subject is administered up to about 90 milligrams of domperidone per day. In some embodiments, a subject is administered up to about 100 milligrams of domperidone per day. In some embodiments, a subject is administered up to about 120 milligrams of domperidone per day. In some embodiments, a subject is administered up to about 140 milligrams of domperidone per day. In some embodiments, a subject is administered up to about 160 milligrams of domperidone per day. In some embodiments, a subject is administered up to about 180 milligrams of domperidone per day. In some embodiments, a subject is administered up to about 200 milligrams of domperidone per day. In some embodiments, a subject is administered up to about 250 milligrams of domperidone per day. In some embodiments, a subject is administered up to about 30 milligrams of domperidone four times per day (up to a total of about 120 milligrams of domperidone per day).

Treatment can be initiated with smaller dosages that are less than the optimum dose of domperidone, followed by an increase in dosage over the course of the treatment until the optimum effect under the circumstances is reached. If needed, the total daily dosage may be divided and administered in portions throughout the day.

For effective treatment of nausea or vomiting, or for effective enhancement of lactation, one skilled in the art may recommend a dosage schedule and dosage amount adequate for the subject being treated. In some embodiments, dosing may occur one to four or more times daily for as long as needed. The dosing may occur less frequently if the compositions are formulated in sustained delivery vehicles. The dosage schedule may also vary depending on the domperidone concentration, which may depend on the needs of the subject.

The compositions of the invention for treating nausea and vomiting, for treating gastrointestinal motility disorders, and for enhancing lactation, can also be co-administered with other well known therapeutic agents that are selected for their particular usefulness against the condition that is being treated. For example, such therapeutic agents can be pain relievers, stomach antacids, proton pump inhibitors (such as omeprazole), histamine H₂ receptor antagonists (such as cimetidine and ranitidine) compounds which lessen untoward effects of the domperidone, or other known agents that can inhibit, suppress, or otherwise treat nausea or vomiting, known agents that treat gastrointestinal motility disorders, or known agents that enhance or promote lactation.

The administration of these additional compounds may be simultaneous with the administration of domperidone composition, or may be administered in tandem, either before or after the administration of the domperidone composition, as necessary. Any suitable protocol may be devised whereby the various compounds to be included in the combination treatment are administered within minutes, hours, days, or weeks of each other. Repeated administration in a cyclic protocol is also contemplated to be within the scope of the present invention.

In the methods of the invention, subjects having nausea or who are vomiting are identified and treated with domperidone formulations as described herein for a sufficient time and with a sufficient amount to alleviate at least one sign or symptom of nausea or vomiting episodes.

The subject can be any animal, and can be a mammal such as a human, mouse, rat, hamster, guinea pig, rabbit, cat, dog, monkey, cow, horse, pig, and the like. In some embodiments, the subject is human.

The following examples are provided to describe the invention in greater detail. They are intended to illustrate, not to limit, the invention.

### Example 1

### Prophetic Example

### Volunteer Study of Domperidone Cardiac Toxicity

This example describes how the inventive domperidone formulations can be clinically evaluated for their potential to cause cardiac arrhythmias

A randomized, placebo and positive-controlled multiple dose trial will take place at a chosen clinic. The study will be conducted over five days, and will include 40-80 healthy subjects, defined as having no known medical problems, a normal baseline ECG, and not presently taking prescription medications. Subjects can be male or female, 18 years of age or older.

To establish a baseline, a placebo will be administered to study subjects four times on a single day, with patients receiving continuous digital ECG monitoring for 24 hours. Over the course of five days, 10, 20, or 30 mg of domperidone transmucosal formulations will be administered to study subjects four times per day. With respect to dosage of domperidone, because it is possible that lower doses of domperidone formulations will be effective, this study may be performed using single doses at any concentration between 1 and 30 mg of domperidone. Blood plasma levels of domperidone will be determined, and continuous ECG monitoring will occur on day five, for 24 hours. As a positive control, one group of study subjects will be administered moxifloxacin for five days, with continuous digital ECG monitoring on day five, for 24 hours.

One purpose of this study will be to demonstrate the pro-arrhythmic risk involving the administration of oral transmucosally-delivered domperidone (“OTD-domperidone”) at a dosing range determined by a previously performed pharmacokinetic study. The variables to be measured by this QT study will be: (1) baseline and 5-day treatment, 24 hour continuous ECG studies; (2) serum levels of domperidone; (3) symptom diary.

### Example 2

### Prophetic Example

### Evaluation of Safety and Efficacy of Domperidone Formulations for Transmucosal Delivery

This example describes how the inventive domperidone formulations can be clinically evaluated for their potential to penetrate mucosal membranes for transmucosal delivery of the active agent, for the treatment of nausea and vomiting, and for the enhancement of breast milk production.

To clinically assess the safety and efficacy of the inventive compositions and methods to administer domperidone transmucosally, the following protocol will be employed. The study will take place at a single center, or multiple centers. The study will evaluate the dose range and safety of OTD-domperidone in patients with diabetic or idiopathic gastroparesis. Additional patient populations to be studied include patients with proximal gastrointestinal motility disorders such as gastroparesis (diabetes, idiopathic or post viral, drug-induced, post-operative), functional dyspepsia, and gastroesophageal reflux, or hypolactation during nursing.

To assess dose ranging, varying doses of OTD-domperidone will be evaluated in a range which will be determined from pharmacokinetic (PK) studies performed prior to this study. The PK study will assess the timing of peak blood levels in normal patients receiving ODT and oral domperidone. Since in vitro permeation data suggests OTD-domperidone permeation through the buccal mucosa is four times better than oral domperidone, it is possible that a dose as low as 2-4 mg of OTD-domperidone four times per day may be effective. These data need to be compared with the usual oral dose of 10-20 mg four times per day. This total reduction in dose allowed by OTD-domperidone of 32-64 mg is expected to significantly reduce the risk of cardiotoxicity. After defining the pharmacologically effective dose range through relying on timing of peak blood levels, a QT study will be performed as defined by FDA standards to demonstrate cardiac safety. Once cardiac safety is demonstrated, the next study will be a dose ranging study to define efficacy and safety of OTD domperidone in patients with gastroparesis. The dose ranging study will be performed in patients with idiopathic or diabetic gastroparesis where various dosing regimens will be compared to various outcomes including: (1) improvement in patient symptoms as determined by the Gastroparesis Cardinal Symptom Index (Revicki et al (2003) Aliment Pharmacol Ther 18:141-50); (2) blood levels of domperidone; (3) frequent ECG monitoring. Dosing may depend on the particular vehicle chosen to deliver the active agent. Dosing regimens of OTD-domperidone may range from as low as 1 mg four times per day to as high as 30 mg four times per day. For example, a tablet will be designed such that 10% of a 30 mg tablet is absorbed quickly through the oral mucosal route, and the other 90% is absorbed intestinally and more slowly.

One dosing regimen based on current in vitro trials is 2-4 mg four times per day, however, timing in relation to meals is still unclear. Dose timing in relation to meals may be quite sooner than traditional per os dosing. This tailoring of dosing as close to meals as possible may be of great convenience to the patient. The oral transmucosal delivery of domperidone may allow for greater specificity of timing of dose prior to a meal more reliable. Patients with gastroparesis may get a more reliable therapeutic effect sooner with OTD-domperidone since slow gastric emptying will not effect buccal absorption of the drug. Slowed and variable absorption of traditional per os domperidone is a problem in patients with delayed gastric emptying.

The study will include 50 to 500 patients. Basic inclusion criteria for the selection of test subjects will be as follows: Subjects may be male or female and of any race and aged 18-75 years. Subjects should present with diabetic or idiopathic gastroparesis symptoms for in excess of three months. Inclusion criteria include: (a) normal upper endoscopy except for the possible presence of food or fluid, performed within the three months prior to enrollment; (b) Gastroparesis Cardinal Symptom Index (GCSI) of greater than 2.5 (Revicki et al. (2003) Aliment. Pharmacol. Ther. 18:141-150); (c) an abnormal solid gastric emptying scan defined by greater than 60% retention at 2 hours, or greater than 10% at 4 hours (Lin et al. (2004) Diabetes Care 27:1071-6). Written informed consent will be obtained from patients prior to participation in the study.

Basic exclusion criteria for the selection of test subjects will be as follows: (a) any structural abnormality of the GI tract; (b) having recently taken any drugs that can slow GI motility; (c) any post-surgical state of the GI tract; (d) pseudo-obstruction syndromes; (e) anything impairing cognitive abilities; (f) primary eating or swallowing disorders; (g) chemical dependency; (h) positive pregnancy test or breast feeding; (i) psychogenic vomiting; (j) allergy to domperidone; (k) baseline QT prolongation; (l) having recently taken any drugs known to prolong QT interval; (m) Hx of prolactin hypersecretion; (n) severe hepatic or renal impairment). In addition, test subjects must not concurrently enroll in a research study or use an investigational drug or device within 30 days prior to enrollment in the study, and throughout the duration of the study. Finally, test subjects may be excluded if they display any condition in which the investigator feels may confound the study results, put the subject at risk, or otherwise may interfere with the subjects' participation in the study.

Study participants will be divided into four groups: (1) OTD-domperidone formulation containing 10 mg domperidone, administered 30 minutes before a meal and 30 minutes before sleep; (2) OTD-domperidone formulation containing 20 mg domperidone, administered 30 minutes before a meal and 30 minutes before sleep; (3) OTD-domperidone formulation containing 30 mg domperidone, administered 30 minutes before a meal and 30 minutes before sleep; (4) Oral domperidone formulation containing 20 mg domperidone, administered 30 minutes before a meal and 30 minutes before sleep.

The study will assess the following variables: (1) Symptom improvement measured within the first hour after drug administration, and after six weeks of treatment as measured by the gastroparesis cardinal symptom index; (2) absorption of domperidone as measured by serum levels of domperidone over time; (3) ECG monitoring.

### Example 3

### Prophetic Example

### Rodent Model for Dosing Domperidone

The minimal dosage of domperidone required to effectively reverse the effect of quinpirole-HCl will be determined by a comparison of gastric emptying measured in the control group and compared to the gastric emptying in a domperidone-treated group. Gastric emptying will be measured according to method of Trudel et al. (2002) [AJP-Gastrointest Liver Physiol 282:G948]. Fifty total rats will be placed in groups of five with the groups being treated as described below:
1) Naïve control
2) Dopamine D2 agonist-quinpirole-HCl (0.3 mg/kg i.p.) plus saline vehicle (i.v.)
3) quinpirole-HCl plus domperidone (100 μg/kg i.v.)
4) quinpirole-HCl plus domperidone (50 μg/kg i.v.)
5) quinpirole-HCl plus domperidone (10 μg/kg i.v.)
   Rats will be predosed with domperidone (or vehicle control i.v.) for 30 minutes. Rats will be dosed as a dopamine agonist such quinpirole at a concentration of 0.3 mg/kg i.p. A 1.5% methylcellulose solution (in distilled H₂O) and ^{99m}Tc (approx 100,000 cpm) will be administered to male, Sprague-Dawley rats, weighing between 200-250 g intragastrically through a stainless steel gavage needle to conscious rats. After receiving a radioactive meal, the animals will be placed in a wire-bottom cage without access to food and water to allow transit of the intragastric content. All rats will be euthanized by CO₂ inhalation 15-min after receiving the meal. The abdomen will be opened via a midline incision; the stomach will be secured with a single silk ligature at the esophageal junction and two parallel ligatures between the pyloric junction and the duodenum. The stomach with the whole length of the small intestine attached will be isolated and arranged on a flat surface graphed from 0 to 140 cm. The total length of the small intestine will be measured. Intestinal segments (10 cm each) will be separated by ligatures starting from the pyloric junction. The stomach and the intestinal sections will be numbered (1, 2, 3 etc. excised starting with the duodenum) and placed in separate test tubes. Radioactivity remaining in the stomach and in the intestinal segments, arranged in proximal to distal order will be measured using a gamma-counter. The data (c.p.m.) will be expressed as percentage of the total recovered radioactivity. Gastric emptying will be measured by the percentage of total recovered radioactivity remaining in the stomach 15-min after the intragastric infusion. The small intestinal transit will be assessed by the geometric center calculated as a function of the amount of intragastric content transported to each segment along the intestine. Calculations will be made according to the method of Miller and coworkers (1981): geometric center=Σ(% of total radioactivity per segment×number of segment)/100. The small intestinal transit will be characterized also by the maximal distance (cm) reached by the head of the meal along the length of the small intestine.

The disclosures of each and every patent, patent application, publication, and accession number cited herein are hereby incorporated by reference in their entirety. While this invention has been disclosed with reference to specific embodiments, it is apparent that other embodiments and variations of this invention may be devised by others skilled in the art without departing from this true scope of this invention. The appended claims are intended to be construed to include all such embodiments and equivalent variations.

1. A pharmaceutical composition comprising from about 1 mg to about 9 mg of domperidone.

2. A pharmaceutical composition of claim 1 wherein the composition comprises from about 2 mg to about 4 mg of domperidone.

3. A pharmaceutical composition of claim 1 wherein the composition comprises a permeation enhancer.

4.-6. (canceled)

7. A pharmaceutical composition of claim 1 further comprising a sustained release agent.

8. A pharmaceutical composition of claim 1 wherein the concentration of domperidone is from about 0.01% to about 90% of the dry matter weight of the composition.

9. (canceled)

10. A pharmaceutical composition of claim 1 wherein domperidone is incorporated into a dissolvable matrix material.

11. An oral transmucosal solid dosage form comprising from about 1 mg to about 9 mg of domperidone.

12. (canceled)

13. An oral transmucosal solid dosage form of claim 11 wherein the oral transmucosal solid dosage form further comprises a permeation enhancer.

14.-16. (canceled)

17. An oral transmucosal solid dosage form of claim 11 wherein the composition further comprises a sustained release agent.

18. An oral transmucosal solid dosage form of claim 11 wherein the concentration of domperidone is from about 0.01% to about 90% of the dry matter weight of the composition.

19. (canceled)

20. An oral transmucosal solid dosage form of claim 11 wherein domperidone is incorporated into a dissolvable matrix material.

21. A method of delivering the pharmaceutical composition of claim 1 into a subject by transmucosal administration.

22. A method of claim 21 wherein the pharmaceutical composition is administered orally, intranasally, vaginally, rectally, transdermally, or is sublingual.

23. A method of claim 21 wherein the pharmaceutical composition of claim 1 is co-administered with at least one other therapeutic agent.

24. (canceled)

25. A method of claim 21 wherein the pharmaceutical composition is administered in a daily dose range of from about 0.01 mg/kg to about 500 mg/kg of domperidone relative to the weight of the subject.

26. A method of claim 21 wherein the subject is administered a total amount of from about 1 mg to about 500 mg of domperidone per day.

27. A method of claim 21 wherein the subject is administered a total amount of up to about 40 mg of domperidone per day.

28. A method of treating nausea, vomiting, or a gastrointestinal motility disorder in a subject by transmucosal administration of a therapeutically effective amount of the pharmaceutical composition of claim 1 to the subject.

29. The method of claim 28 wherein the pharmaceutical composition is administered intranasally, vaginally, rectally, orally, transdermally, or is sublingual.

30. (canceled)

31. A method of claim 28 wherein the pharmaceutical composition is co-administered with at least one other therapeutic agent.

32. (canceled)

33. A method of claim 28 wherein the pharmaceutical composition is administered in a daily dose range of from about 0.01 mg/kg to about 500 mg/kg of domperidone relative to the weight of the subject.

34. A method of claim 28 wherein the subject is administered a total amount of from about 1 mg to about 500 mg of domperidone per day.

35. A method of claim 34 wherein the subject is administered a total amount of up to about 40 mg of domperidone per day.

36.-43. (canceled)

44. A method of enhancing lactation in a subject by transmucosal administration of a therapeutically effective amount of the pharmaceutical composition of claim 1 to the subject.

45. A method of claim 44 wherein the pharmaceutical composition is administered intranasally, vaginally, rectally, orally, transdermally, or is sublingual.

46. (canceled)

47. A method of claim 44 wherein the pharmaceutical composition is co-administered with at least one other therapeutic agent.

48. (canceled)

49. A method of claim 44 wherein the pharmaceutical composition is administered in a daily dose range of from about 0.01 mg/kg to about 500 mg/kg of domperidone relative to the weight of the subject.

50. A method of claim 44 wherein the subject is administered a total amount of from about 1 mg to about 500 mg of domperidone per day.

51. A method of claim 50 wherein the subject is administered a total amount of up to about 40 mg of domperidone per day.
